(19) United States
(12) Patent Application Publication
(10) Pub. No.: US 2004/0213768 A1
(43) Pub. Date: Oct. 28, 2004

(54) Preparation for biotransplantation and xenotransplantion and uses thereof

(76) Inventors: Robert Bartlett Elliott, Auckland (NZ); Stephen John Martin Skinner, Auckland (NZ); Christopher Edward Williams, Auckland (NZ)

Correspondence Address: YOUNG & THOMPSON, 745 SOUTH 23RD STREET 2ND FLOOR, ARLINGTON, VA, 22202

(21) Appl. No.: 10/757,428

(22) Filed: Jan. 15, 2004

Related U.S. Application Data
(63) Continuation-in-part of application No. 09/959,560, filed on Oct. 30, 2001.
Application No. 09/959,560 is a 371 of international application No. PCT/NZ00/00064, filed on Apr. 28, 2000.

(30) Foreign Application Priority Data
Apr. 30, 1999 (NZ) 335553

(51) Int. Cl. A61K 48/00

(52) U.S. Cl. 424/93.21; 424/93.7

(57) ABSTRACT

Method of preparing and administering a treatment species to a mammalian recipient, the treatment species including one or more of:

a) a cell population capable of producing one or more factors, or

b) a cell culture capable of producing one or more factors or

c) conditioned media from a cell culture containing one or more factors.

Preferably the treatment species is capable of releasing or administering to the recipient, a secretion derived from the cells, more preferably the secretion includes cell derived factors.

### CROSS REFERENCE TO RELATED APPLICATIONS

This application is a continuation-in-part of co-pending application no. 09/959,560, filed on Oct. 30, 2001. Application no. 09/959,560 is the national phase of pct international application no. PCT/NZ00/00064 filed on Apr. 28, 2000 under 35 U.S.C. § 371. The entire contents of each of the above-identified applications are hereby incorporated by reference.

### TECHNICAL FIELD

The present invention relates to treatment preparations suitable for treatment of cells of a mammalian recipient, as well as treatment of the recipient by administration of the preparation. More particularly but not exclusively it relates to treatment preparations comprising or including cell-derived factors and/or cells capable of producing or secreting such factors.

### BACKGROUND

Background and Rationale for Biotransplants.

Strategies of treatment aimed at neurodegenerative diseases of the central nervous system (CNS) as well as acute injury have been largely unsuccessful in treating injury to the brain. A major obstacle to the development and clinical use of such therapies is a difficulty in transporting neuroprotective drugs into the nervous system, particularly the CNS. The presence of the blood-brain barrier (BBB) makes administration of neuroprotective compounds difficult as it provides a unique physical and enzymatic barrier that segregates the brain from the systemic circulation.

The BBB has two major parts; endothelial (blood-brain), and ependymal (blood-cerebrospinal fluid (CSF)) comprised of astrocytes, neurons, pericytes, microglial and leukocytes from the general circulation. These barriers consist of tight junctions that connect the cerebrovascular cells and prevent the diffusion between cells. The B-CSF barrier is located at the circumventricular organs (CVOs) and is formed by tight junctions between ependymal cells.

The main CVO is the CSF secreting choroid plexus that lines the lateral ventricles, the roof of the ventricle, and the fourth ventricle. Unlike the BBB, the capillary ependymal cells of the CVOs are fenestrated, allowing molecules to “leak through”. Cerebrospinal fluid fills the four ventricles, and circulates around the spinal cord and over the convexity of the brain, where the CSF is absorbed into the superior sagittal sinus by way of specialised processes in the meninges (e.g., the arachnoid granulations). The CSF is continuous with the brain interstitial (extracellular) fluid, and solutes, including macromolecules, are exchanged freely between CSF and interstitial fluid.

Once in the cerebral ventricles, molecules of all sizes can diffuse through a single porous ependymal cell layer, and enter the continuous interstitial fluid network that bathes the brain. Because of this, direct administration of therapeutic compounds into the brain has been achieved by implantation or administration of the therapeutic substance directly into the ventricles of the brain via intracerebroventricular administration. In addition, numerous patents, such as U.S. Pat. No. 5,573,528 and U.S. Pat. No. 5,853,385, describe the implantation of implants directly into the tissue of the brain.

Within the patent literature, there are a number of patents that have been filed which describe the implantation o f both live cells and manufactured slow release formulations into specific parts of the CNS, such as U.S. Pat. No. 5,853,385. In addition, there are many patents which describe the administration of compounds via intracerebroventricular administration. The difficulty with these modes of administration is that these are very invasive techniques with a number of associated risks, such as damage to the surrounding tissue by the needle or implement used to administer the substance. In addition, every time a needle passes through the brain tissue there is a 3% chance of a significant bleeding episode. This limits the numbers of penetrations during single and multiple operative episodes.

The problem to be solved is to identify an effective treatment strategy for at least one nervous system condition (whether already existing or in the future).

### OBJECT OF THE INVENTION

It is an object of the present invention to provide a method and means for treatment of the nervous system, in particular the brain, or at least to provide the public with a useful choice.

### BRIEF DESCRIPTION OF THE INVENTION

In a first aspect of the invention, the invention provides a treatment species for administration to a mammalian recipient comprising or including one or more of:

a) a cell population capable of producing one or more factors, or

b) a cell culture capable of producing one or more factors or

c) conditioned media from a cell culture containing one or more factors.

Preferably the treatment species is capable of releasing or administering to the recipient, a secretion derived from the cells, more preferably the secretion includes cell derived factors.

Preferably the factors are neurotrophins, growth factors, matrix cell support factors, proteases capable of degrading toxic protein precipitates (such as amyloid and huntingtin), and proteins capable of complexing toxic metal ions (such transferrin and ceruloplasmin).

Preferably the cells are derived from the embryonic neural crest.

Preferably the cells are selected from one or more choroid plexus cells or one or more glial or glial-derived cells or epithelial cells.

Additionally or optionally these cells may have been subject to genetic modification.

Preferably the cells are choroid plexus cells and the treatment species is capable of releasing or administering to the recipient, a choroid plexus derived secretion, more preferably the secretion includes choroid plexus derived factors.

Preferably the cells are living cells.

Preferably the treatment species is derived from one or more choroid plexus cells obtained or derived from a donor mammalian species. Preferably or alternatively the treatment species is derived from a cell culture, which may be a primary culture and/or a secondary culture and/or cell lines derived from choroid plexus cells including immortalized cell lines.

In one embodiment the donor mammalian species is a different species from the recipient. Preferably the donor mammalian species is a pig, rabbit or rat; more preferably it is a virus-free neonatal pig, rabbit or rat.

In a second embodiment the donor mammalian species is human, as is the recipient (who may or may not also be the donor).

In one embodiment the treatment species may comprise or include:

one or more choroid plexus cells.

In one embodiment the treatment species may comprise or include:

one or more choroid plexus cells encapsulated in a suitable encapsulation medium.

Preferably the encapsulation medium is an alginate.

In one embodiment the treatment species may comprise or include:

one or more naked choroid plexus cells.

In one embodiment the treatment species may comprise or include:

one or more choroid plexus cells contained within a confinement means.

Preferably the confinement means is factor permeable in vivo.

In one embodiment the treatment species may comprise or include:

one or more isolated choroid plexus cells.

In one embodiment the treatment species may comprise or include:

media harvested from choroid plexus cells (whether these be naked, isolated, cultured, modified or otherwise).

In one embodiment the treatment species may comprise or include:

one or more choroid plexus cells and/or the media harvested from one or more choroid plexus cells (whether these be naked, isolated, cultured, modified or otherwise), in a pump or implantable infusion device.

In one embodiment the treatment species may comprise or include:

one or more choroid plexus cells and/or the media harvested from one or more choroid plexus cells (whether these be naked, isolated, cultured, modified or otherwise), in a bio-erodable polymer.

In one embodiment the treatment species may comprise or include:

cerebrospinal fluid containing one or more choroid plexus cells and/or the secretion from one or more choroid plexus cells obtained from the recipient, or another mammalian species.

According to a further aspect of the invention there is provided a method of preparing a treatment species for administration to a recipient mammal comprising or including the steps of:

a) obtaining one or more cells capable of producing one or more factors, from a donor species

b) preparing the treatment species.

Preferably the one or more cells are choroid plexus cells.

Preferably the method comprises or includes the steps:

1) obtaining one or more choroid plexus cells from a donor species;

2) culturing the one or more choroid plexus cells;

3) preparing the treatment species.

Preferably step 1) of obtaining the one or more choroid plexus cells from the donor species comprises or includes obtaining the fresh tissue from the donor and dissociating the tissue mechanically and preferably by Liberase (Roche) digestion.

Preferably step 2) of culturing the one or more choroid plexus cells comprises or includes preparing the cells in such a way as to produce choroid plexus cell clusters of a regular size (preferably between 50-300 microns in diameter).

Preferably the step 3) of preparing the treatment species may comprise or include one of the following:

encapsulation of the cells, or media obtained therefrom in a suitable encapsulation medium, more preferably in alginate.

confinement of the cells, or media obtained therefrom in a suitable confinement means,

housing of the cells, or media obtained therefrom in a pump or implantable infusion device;

housing of the cells, or media obtained therefrom in a bioerodable polymer;

addition of the cells or media obtained therefrom to a pharmaceutically acceptable diluent and/or excipient and/or carrier.

According to a further aspect of the invention there is provided a treatment species prepared according to the above method.

According to a further aspect of the invention there is provided a method of administering a treatment species to a recipient comprising or including:

preparation of a treatment species as previously described,

administering the treatment species to a targeted area of the recipient.

Preferably the administration of the treatment species to the recipient results in one or more of the following events:

treatment of cells of the nervous system damaged by events such as injury, disease, trauma.

protection against damage to cells of the nervous system arising from future events as injury, disease, trauma.

prevention or minimisation of apoptotic nervous system cell death.

regeneration of damaged cells of the nervous system.

impeding or stopping cell death cascades resulting from events such as nervous cell injury, disease, trauma.

Preferably the cells are in the central nervous system; alternatively the cells are in the peripheral nervous system; most preferably the cells are in the brain.

Preferably the administration of the treatment species results in one or more of the following:

treatment or prevention of a neurodegenerative disease;

repair of damage caused by acute trauma to the brain;

treatment of damage resulting from pre-birth asphyxia;

treatment of damage resulting from neonatal ischemia (pre, during, post birth);

treatment of infection related cell death (including from meningitis and encephalitis);

treatment of damage resulting from pressure related cell death (such as resulting from head injury to the recipient);

treatment of auto-immune disorders, including for example, demyelinating conditions (such as multiple sclerosis); rheumatoid arthritis, crohn's disease, ulcerative colitis;

Treatment of sense loss due to apoptotic events, such as RP, diabetic retinopathy, macular degeneration, optic nerve damage;

Treatment of inborn errors of metabolism that mostly affect the central nervous system.

Preferably the step of administering the treatment species to a targeted area of the recipient includes one or more of the following:

administering the treatment species into the central nervous system;

administering the treatment species to a region outside but adjacent or proximal the central nervous system;

administering the treatment species directly into the region of the recipient which has suffered damage;

administering the treatment species to a region outside but adjacent or proximal the region of the recipient which has suffered damage;

administration of the treatment species into the brain parenchyma;

administration of the treatment species into the recipient so as to selectively target apoptotic cells; more preferably this comprises

administering the treatment species into the margin of the damaged region;

administration of the treatment species into the recipient so as to selectively target necrotic cells; more preferably this comprises administering the treatment species into the central aspect of the damaged region;

administration of the treatment species into the ventricle;

administration of the treatment species via lumbar puncture;

administration of the treatment species into a CSF containing region.

Preferably the step of administering the treatment species to a targeted area of the recipient comprises or includes any administration so as to expose the targeted area to choroid plexus derived secretion, preferably the secretion includes choroid plexus derived factors.

Preferably the step of administering the treatment species to a targeted area of the recipient comprises or includes one or more of:

administration resulting in substantially immediate delivery of the treatment species to a targeted area; or

administration resulting in controlled delivery of the treatment species to the targeted area over a pre-selected time period; preferably the pre-selected time period is greater than five minutes;

Preferably the method includes one or more steps of:

suppressing the immune response of the recipient, more preferably by administration of immunosuppressive agents or drugs;

cooling the recipient;

Administering the cell preparation via a cannulated blood vessel.

According to a further aspect the present invention consists of a method of preventing, treating and/or ameliorating a neurological injury, disease or imbalance, comprising or including:

1) preparing an implant

2) implanting the implant in or around the central nervous system

wherein said implant results in, directly or indirectly, a beneficial effect on said neurological injury.

According to one embodiment the implant consists of conditioned media.

According to a second embodiment the implant consists of living cells formed from the group of cells including choroid plexus cells, glial derived cells and neurons. Preferably said living cells are formed from a homogeneous mix of cell populations.

Preferably the cells are encapsulated in a biocompatible medium.

Preferably the implant is implantable into a localised area in or around the central nervous system proximate to the neurological injury or alternatively into supporting structures of the central nervous system.

According to a further aspect of the invention there is provided an implant for implantation into the central nervous system and/or surrounding supporting structures of a mammalian recipient,

wherein the said implant consists of conditioned media and/or living cells formed from a homogeneous mix of cell populations.

Preferably said implant consists of one or more living cells formed from a group of cells including choroid plexus cells, glial derived cells and neurons.

According to a further aspect the present invention provides a surgical method for treatment of a human comprising or consisting the following steps:

1. accessing through the skull and dura mater;

2. administering an implant as described previously into a cerebral fluid filled space.

In one embodiment step 2 comprises or includes administering the implant directly into the brain parenchyma.

In an alternative embodiment, step 2 comprises or includes administering the implant external to the brain parenchyma. Preferably the implant is located subdurally but still external to the brain parenchyma.

In a further aspect, this present invention consists of a pharmaceutical composition for treatment or prevention of a disease or condition in a mammal in need of treatment by therapeutic administration of an implant comprising:

1. one or more living cells capable of producing one or more factors;

2. at least one permeation-enhancement agent for transmucosal drug uptake.

Preferably the one or more living cells include one or more choroid plexus cells.

DEFINITIONS

| | |
|---|---|
| “Damage” | includes damage to a cell including physical damage, |
| | disruption to or impairment of, normal cellular function, |
| | including damage as a result of neurological injury or |
| | disease or trauma or disorder. It also includes any cell |
| | which is adversely affected by injury, disease, imbalance or |
| | other adverse event. |
| “Nervous System” | includes cells of the peripheral and central nervous system. |
| “brain” | refers to the portion of the central nervous system as |
| | distinct form the spinal cord that is made up of white and |
| | grey matter which are formed form neurons and glial |
| | derived cells. |
| “Central Nervous System” | refers to both the brain and spinal cord. |
| “Meninges” | refers to the structures surrounding the brain as made up of |
| | the dura mater, the arachnoid, and the pia mater. |
| A “neurological disease” | covers any disorder of the central nervous system. It may |
| | for example be a global neurodegenerative disease, such as |
| | ageing, vascular disease, Alzheimer's disease, or the more |
| | localised Parkinson's disease, or the autoimmune disease |
| | multiple sclerosis (MS), it may be a result of an injury, such |
| | as a stroke, anoxia/asphyxia, or physical injury such as |
| | from a blow to the head, it may be a result of exposure to |
| | local (eg meningitis) or systemic toxins, and it may be |
| | neoplastic. It may be genetically based, such as |
| | Huntington's chorea, or a disorder of metabolism such as |
| | lysosomal storage disease. There is a group of “global |
| | neurodegenerative diseases” including AZ and others, |
| | affecting the elderly, the usual pattern of response to acute |
| | injury (such as ischaemia), affecting any age group |
| | including stroke victims and car accident victims, |
| | autoimmune diseases such as MS, PD, and certain diseases, |
| | including deficiencies of metabolism, of neonates and |
| | fetuses. Indeed PD may be more global than is currently |
| | appreciated. The known defects in and around the basal |
| | ganglia may be reflected elsewhere. |
| “restorative effect” | includes any beneficial modification of the disease process, |
| | including palliative, restorative, or proliferative effects |
| | acting on neural tissue, glia, or vascular elements. We tend |
| | to use “trophic” and “growth” factors interchangeably. |
| “rejuvenation” | means attempts to reverse changes in a brain commonly |
| | considered to be the usual, if not the normal consequences |
| | of ageing, such as loss of volume, loss or atrophy of |
| | neurones, loss of memory, and loss of ability to cope with |
| | complex sensory inputs. Rejuvenation could also comprise |
| | restorative effects on existing neurones, neural rescue as |
| | required after an asphyxic episode, or “sick neurones”. |
| A “neurological injury” | includes injury to the central nervous system and/or |
| | imbalance as caused by such injury resulting from stroke, |
| | acute trauma and infection. |
| “factor” | any substance having a beneficial effect on a cell. |
| “neurotrophin” | is a subset of “factor”. It includes entities with related |
| | structures that are known to support the survival of neurons. |
| | They include species such as growth factors. |

### BRIEF DESCRIPTIONS OF THE DRAWINGS

The invention will now be described with reference to the Figures in which:

FIG. 1 is a schematic diagram showing the layers of the meninges and the cerebral cortex;

FIG. 2 is a schematic diagram showing the central nervous system and the supporting structures including the meninges;

FIG. 3 is a graph depicting stroke-induced motor deficits in stroke-only control animals (∘), stroke animals administered control transplant (▪), and stroke animals administered choroid plexus transplants (▴), wherein *=P<0.01;

FIG. 4 is a graph depicting the neurologic impairment as assessed by the Bederson Test observed in stroke-only control animals (▪), stroke animals administered control transplant (□), and stroke animals administered choroid plexus transplants ( [symbol] ), wherein *=P<0.0001;

FIG. 5 is a graph depicting the mean striatal infarct volume observed in stroke-only control animals (▪), stroke animals administered control transplant (□), and stroke animals administered choroid plexus transplants ( [symbol] ), wherein *=P<0.05; and

FIG. 6 is a graph depicting the effect of conditioned media from cultured choroid plexus on neuronal cell viability, wherein *=P<0.0001 versus 0%; **=P<0.0001 versus 0%, 1% and 3%.

### DETAILED DESCRIPTION OF THE INVENTION

The present invention deals with the preparation of a treatment species which incorporates factors derived from therapeutic cells, and/or therapeutic cells, taken from a donor. It also deals with administration of the treatment species into a recipient to treat cells of the nervous system of the recipient (be these cells damaged or yet to be damaged).

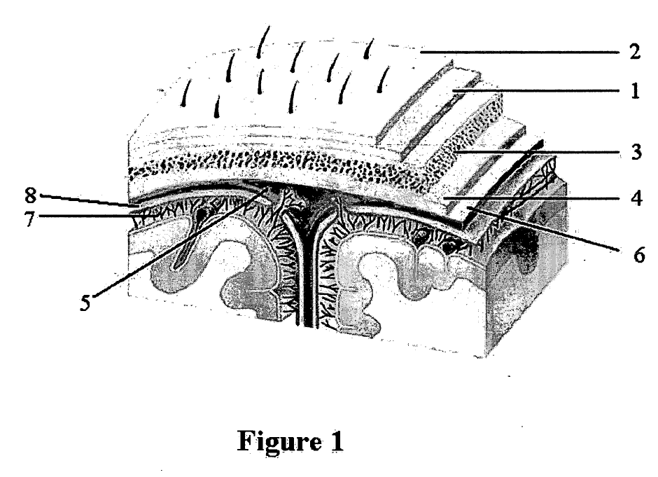

The present invention recognises that there are many avenues of administration of treatment species derived from the therapeutic cells which may be beneficial to the recipient. With reference to FIG. 1 there is illustrated a section of the human brain with external structures. It shows the skull 1, the skin 2, the dura 3, the arachnoid layer 4, the arachnoid granulation, 5, brain 6, the arachnoid space 7 and the subdural space 8.

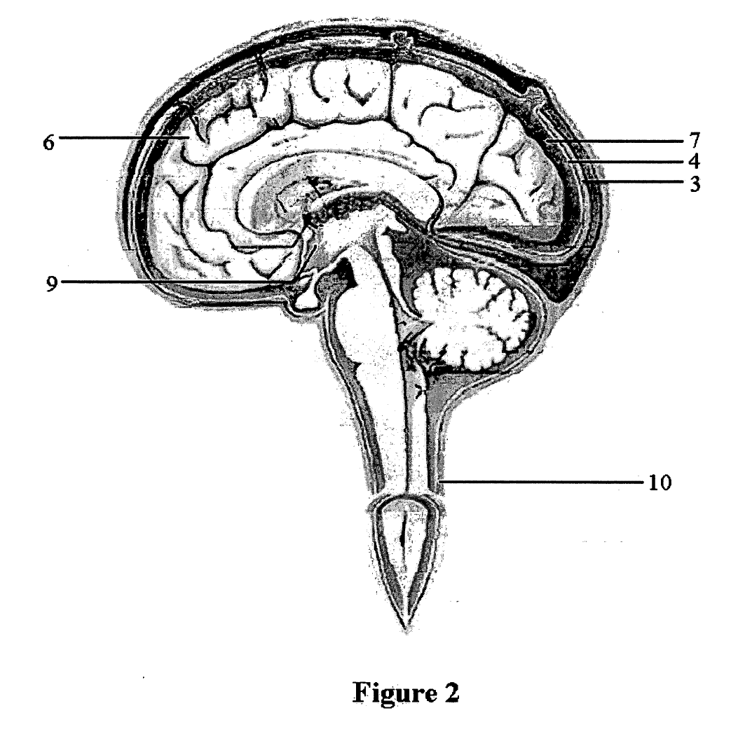

FIG. 2 illustrates a sagittal section of the central nervous system and surrounding structures. This shows the central nervous system as comprised of the brain 6 and spinal cord 10. In addition this Figure illustrates the surrounding meninges with structures as show in FIG. 1. In addition FIG. 2 illustrates a ventricle of the brain 9 (in this case the third ventricle).

The avenues of administration within the invention can include any CSF filled space such as the ventricle 9 or within the meninges of the brain (together shown by 3, 4 7, 8). One such possible site of administration outside of the brain is shown in FIGS. 1 and 2 as the sub-arachnoid space 7 or the subdural space 8, which is a space below the dura 3 that is enlarged after injury. The means of delivery device and location of the delivery device in the recipient all have important roles to play in the treatment.

We have previously filed a PCT patent application, published as WO 00/66188, the contents of which are incorporated herein in their entirety. This deals with certain aspects of use of choroid plexus cells to prepare treatment species. It also deals with administration into the brain. With reference to FIG. 2 the administration of the choroid plexus cells in WO 00/66188 were into the ventricle 9.

The present invention recognises that certain cells (therapeutic cells) from a donor are capable of excreting factors, such as neurotrophins and other therapeutic substances. These cells, when dealt with according to the invention, and the factors or other secreted entities from the cells, can be used to treat damaged (which hereafter also includes yet to be damaged) cells in the recipient.

The preferred excretory cells dealt with in this invention include choroid plexus cells and also glial or glial-derived cells. However, as will be envisaged by one skilled in the art, there may be other suitable cells also effective in a recipient such as islets of Langerhans, neural progenitor cells, immune stem cells.

In the case of the choroid plexus cells the resultant treatment species which will incorporate such cells or cell derivatives, are capable of releasing or administering to the recipient, a choroid plexus derived secretion which includes choroid plexus derived factors.

The choroid plexus has been associated with the production of CSF and the formation of the CSF-blood barrier (Aleshire S L et al., “Choroid plexus as a barrier to immunoglobulin delivery into cerebrospinal fluid.” J Neurosurg. 63:593-7, 1985). However, its broader function is the establishment and maintenance of baseline levels of the extracellular milleu throughout the brain and spinal cord, in part by secreting a wide range of growth factors into the CSF. Studies have confirmed the presence of numerous potent trophic factors within choroid plexus including TGFb, GDF-15, GDNF, IGF2, NGF, NT-3, NT-4, BDNF, VEGF, and FGF2 (for review see Johanson C E et al., “Choroid plexus recovery after transient forebrain ischemia: role of growth factors and other repair mechanisms.” Cell Mol Neurobiol. 20:197-216, 2000).

The present invention recognises the endogenous role of choroid plexus in growth factor production to provide stable and dose controlled protein delivery. This delivery can be modulated, for example by varying the numbers of cells implanted.

The present invention further recognises that choroid plexus cells are potential neural precursor cells in the adult mammalian brain, providing a source of transplantable progenitor cells for cell based therapeutic applications.

The present invention deals with giving rise to the correct environment in the recipient to allow the secreted cocktail from the therapeutic cells to act upon the damaged recipient cells.

1. Source of the Therapeutic Cells

The invention contemplates deriving donor cells from any of the following (which are included in the scope of the invention):

Any mammal including pigs and rats—including isolated or cultured cells as well as those transplanted from a host to a recipient;

The recipient human or another human—including isolated or cultured cells as well as those transplanted from a host to a recipient;

Any genetically modified cell which has the characteristics of a therapeutic cell as described herein.

2. Preparation of the Cells for use as/in the Treatment Species

The cells obtained as set out above may be handled in a number of ways for use in the ultimate treatment species or delivery means.

This includes no additional treatment, with direct and rapid transplantation of the cells into the recipient. It also includes culturing, including:

primary cultured cells derived from any of the above, including those which are frozen, grown in a variety of media as would be envisaged by one skilled in the art;

cell lines derived from any of the above, including those which are frozen, grown in a variety of media as would be envisaged by one skilled in the art.

It further includes application of genetic modification techniques to extracted cells.

It further includes use of functionally enhancing media, including growth media.

3. Treatment Species

The treatment species of the invention is, in the preferred embodiment, that species which is implanted or otherwise administered to the recipient. It is derived in one form or another, from any of the cells discussed in 1 and 2 above. For example, the treatment species may be an extracted/isolated cell, or may comprise therapeutic cells subjected to one or more preparatory steps prior to inclusion in or formation of the treatment species. In another example, the treatment species may comprise one or more factors derived from the cells discussed above.

The following examples are embodiments of the invention, but the invention is not restricted to these examples.

a) “Naked” Cells

In this instance the treatment species administered is simply any therapeutic cell in its natural state following harvesting.

b) Encapsulated cells

This form of treatment species relies upon the incorporation of therapeutic cells of t he invention within capsules. The preferred capsule medium is a biocompatible alginate.

c) Confined Cells

This form of treatment species relies upon the confinement of the cells within a confinement means, such as for example, a tube.

d) Enhanced Media-containing Factors from the therapeutic cells

This form of treatment species comprises media comprising the factors produced by therapeutic cells, as distinct from the cells themselves. This can be obtained in a number of ways. In a preferred embodiment this may involve growing the therapeutic cell of interest as would be known in the art, in a culture media. This process allows the cells to excrete factors and other secreted species into the media to provide an enhanced media. The enhanced media may then be separated from the cells for preparation of the treatment species.

Alternatively, it may be that the cells in the media are simply quiescent, requiring some activating process or factor which will bring about the secretion into the media, to give the enhanced media.

The media may comprise the treatment species, or alternatively may be incorporated within any other treatment species within the invention.

e) Implantable infusion device

This may include a device having a semi-permeable or permeable membrane (or equivalent) providing the factors obtained from the therapeutic cells or enhanced media from such cells access to damaged cells of the recipient. An implantable infusion device may also be prepared by the in situ formation of an active agent containing solid matrix as disclosed in U.S. Pat. No. 6120789. Implantable infusion devices may be passive or active. An active implantable infusion device may comprise an active agent reservoir, a means of allowing the active agent to exit the reservoir, for example a permeable membrane, and a driving force to propel the active agent from the reservoir. Such an active implantable infusion device may additionally be activated by an extrinsic signal, such as that disclosed in WO 02/45779, wherein the implantable infusion device comprises a system configured to deliver the active agent comprising an external activation unit operable by a user to request activation of the implantable infusion device, including a controller to reject such a request prior to the expiration of a lockout interval. Examples of an active implantable infusion device include implantable drug pumps. Implantable drug pumps include, for example, miniature, computerized, programmable, refillable drug delivery systems with an attached catheter that inserts into a target organ system, usually the spinal cord or a vessel. See Medtronic Inc. Publications: UC9603124EN N P-2687, 1997; UC199503941b EN NP-2347 182577-101,2000; UC199801017a EN NP3273a 182600-101, 2000; UC200002512 EN NP4050, 2000; UC199900546bEN NP- 3678EN, 2000. Minneapolis, Minn: Medtronic Inc; 1997-2000.

Such a device may operate simply as a result of being located in the recipient; or alternatively it may operate as a result of some triggering event. Triggering events could include for example, an accident, manual or automatic triggering.

f) Bioerodable Polymer

The therapeutic cells and/or treatment species may, for instance, be embedded into a polymer or other biocompatible substance matrix. This matrix may solubilise or otherwise degrade in vivo thereby allowing release of the cells or the factors derived from the cells into the recipient.

g) Cerebrospinal Fluid CSF

CSF may be obtained from a number of sources. It may be obtained from the recipient themselves or an appropriate mammalian donor, or alternatively may be manufactured or prepared artificially. CSF itself may comprise the treatment species, allowing release or access of therapeutic factors derived from the cells into the recipient. It may supplement or replace the recipient's CSF.

4. Administration/Implantation Modes

The present invention includes modes by which the treatment species can be administered to the recipient. These modes include administration of the treatment species into the brain or external to the brain. With reference to FIGS. 1 and 2 administration of a treatment species can be can be made directly into an area of the brain 6, or into a ventricle 9. Alternatively, extra-brain administration could be made into the subarachnoid space 7 or subdural space 8 which surrounds both the brain and spinal cord.

5. Conditions to be Treated

A wide variety of conditions may be treated by the methods and treatment species of the invention. These include conditions in which cells of the recipient's nervous system would benefit by direct or indirect exposure to the secretion of the therapeutic cells of the invention. It also includes mechanisms by which exposure of the secretion of the therapeutic cells has some downstream beneficial result or effect.

The conditions to be treated include:

neurological diseases such as those previously discussed, including global neurodegenerative diseases such as aging, vascular disease, Alzheimer's; or the more local disease of Parkinson's; autoimmune disorders such as MS; Huntington's disease, inborn errors of metabolism such as Menkes Kinky Hair Syndrome, Wilsons Disease, and other neurological diseases or disorders.

injury to the nervous system, particularly the brain, such as pressure resulting in head injury, stroke, anoxia/asphyxia, and injury resulting from CO₂ or CO poisoning.

6. Treatment Regimes

a) Transplantation

b) Use of Naked therapeutic cells with Immunosuppression

The production of therapeutic cells without encapsulation is quicker, and may be an option in situations where encapsulation technology, or cryo-preserved encapsulated cells are not immediately available.

Alternatively for short-term treatments, for example treatment periods of about one to two weeks, such as for stroke, this may be a preferred option.

The patient will be treated with a range of immunosuppressive agents before, or at the time of, transplantation with the therapeutic cells such as choroid plexus cells. These immunosuppressive agents will prevent or reduce complement-mediated rejection and aspects of cell-mediated responses to the allo- or xeno-transplant. The choroid plexus cells can then be inserted into defined sites with, for example, a fine, narrow-lumen catheter.

c) Catheter delivery

d) Pump

A pump, able to transport the therapeutic cells or factors derived therefrom to the damaged region, or proximal to the region or any other location which may result in a benefit to the damaged region or cells susceptible to such damage, may be located in the body, including in the brain, or elsewhere (external to the body). As described above, this includes active implantable infusion devices.

7. Example 1

### METHODS

All procedures used in this study adhered to NIH and Society for Neuroscience guidelines for use of animals in research. All surgical procedures were conducted under aseptic conditions. All efforts were made to minimize animal suffering and to reduce the number of animals used.

Animals

Adult male Wistar rats (supplied by University of Auckland, NZ) approximately 3 months of age and weighing 250-350 grams served as subjects. Animals were housed in a temperature (22+/−1° C.) and humidity (50+/−5%) controlled environment and had free access to food and water throughout the study, except for 4 hours prior to surgery.

Isolation, Culture, and Encapsulation of Pig Choroid Plexus Cells

Neonatal pigs (strain, sex, age and weight needed) were anaesthetized with ketamine (500 mg/kg) and xylazine (0.15 mg/kg) and killed by exsanguination. The brain was immediately removed and dissected through the midline to reveal the fork of the choroid vessels. The choroid plexus was extracted and placed in Hanks Balanced Salt Solution (HBSS, 0-4° C.) supplemented with 2% human serum albumin. The tissue was chopped finely with scissors, allowed to settle and the supernatant removed. Collagenase (Liberase, Roche, 1.5 mg/ml, in 5 ml HBSS at 0-4° C.) was added and the chopped tissues mixed, allowed to sediment at unit gravity (1×g) and the supernatant was again removed. Collagenase (1.5 mg/ml, in 15 ml HBSS at 0-4° C.) was added and the preparation warmed to 37° C. and stirred for 15-20 minutes. The digested material was triturated gently with a 2 ml plastic Pasteur pipette and passed through a 200 um stainless steel filter.

The resulting neonatal pig preparations were mixed with an equal volume of RPMI medium supplemented with 10% neonatal porcine serum (prepared at Diatranz/LCT). The preparations were centrifuged (500 rpm, 4° C. for 5 minutes), the supernatant removed and the pellet gently re-suspended in 30 ml RPMI supplemented with serum. This procedure produced a mixture of epithelioid leaflets or clusters of cells, about 50-200 microns in diameter, and blood cells. Blood cells were removed by allowing the mixture to sediment at unit gravity for 25 minutes at 0-4° C., removing the supernatant and re-suspending. The preparation was adjusted to approximately 30,000 clusters/ml in RPMI with 10% serum and placed in non-adherent Petri dishes. Half of the media was removed and replaced with fresh media (5 ml) after 24 hours and again after 48 hours. By this time, most clusters assumed a spherical, ovoid or branched appearance.

Prior to encapsulation the cell clusters were washed by sedimenting 3×in 2% human serum albumin (30 ml) at room temperature. The cells were then encapsulated in alginate according to previous published protocols (Elliott et al, CT 2000, 9:895-901; Calafiori et al., Transpl. Proc. 1997, 29:2126-7). Conditioned media was removed after 48 hours and stored at −20° C. for in vitro testing (see below). Encapsulated cells were maintained in culture for xx days prior to transplantation.

Stroke Surgery

Rats were anesthetized using equithesin (300 mg/kg i.p.). Permanent unilateral focal neocortical ischemia was produced using a well-established middle cerebral artery (MCA) occlusion/reperfusion model. Based on our previous studies and those of several other laboratories (American Heart Association. “Stroke Statistics.” 2002. ; Borlongan C. V., Cahill D. W., Sanberg P. R. “Locomotor and passive avoidance deficits following occlusion of the middle cerbral artery.” Physiol Behav. 58:909-17, 1995a.; Borlongan C. V., Sanberg P. R. “Elevated body swing test: a new behavioral parameter for rats with 6-hydroxydopamine-induced hemiparkinsonism.” J Neurosci. 15:5372-8, 1995c), a one-hour occlusion of the MCA was used to produce a maximal infarction. Breifly, an incision was made to expose the right MCA and a nylon suture (length=15-17 mm; tip diameter=24-26 gauge was inserted to completely occlude the MCA (American Heart Association. “Stroke Statistics.” 2002. ; Borlongan C. V., Cahill D. W., Sanberg P. R. “Locomotor and passive avoidance deficits following occlusion of the middle cerbral artery.” Physiol Behav. 58:909-17, 1995a.; Borlongan C. V., Sanberg P. R. “Elevated body swing test: a new behavioral parameter for rats with 6-hydroxydopamine-induced hemiparkinsonism.” J Neurosci. 15:5372-8, 1995c). After a one hour occlusion, the suture was removed and the incision closed using routine procedures. Based on our experience with the MCA occlusion model, body temperature and blood gases of animals undergoing such surgical procedure remain within normal limits (Borlongan C. V., Tajima Y., Trojanowski J. Q., Lee V. M., Sanberg P. R. “Transplantation of cryopreserved human embryonal carcinoma-derived neurons (NT2N cells) promotes functional recovery in ischemic rats.” Exp Neurol. 149:310-21, 1998. ; Borlongan C. V., Yamamoto M., Takei N., Kumazaki M., Ungsuparkom C., Hida H., Sanberg P. R., Nishino H., “Glial survival as enhanced during melatonin-induced neuroprotection against cerebral ischemia.” FASEB J. 14:1307-17, 2000).

Transplantation Surgery

Immediately following MCA occlusion (i.e. within 10 minutes) animals were placed in a stereotaxic apparatus (Kopf Instruments). A craniotomy (2 mm wide×3 mm in length) was performed over the predicted core of the cerebral infarction using a surgical microdrill. The coordinates for the craniotomy were: ML=3.0 mm to 5.0 mm and AP=+1.0 mm to −2.0 mm-from Bregma (Paxinos, G. and C. Watson C. “The Rat Brain in Stereotaxic Coordinates”, Academic Press, New York, 1986). For transplantation, the dura was excised and 50-55 hand-picked microcapsules were suspended in 30 ul of isotonic saline and placed into the previously formed craniotomy. The excess saline was gently removed resulting in a bed of alginate capsules overlying the cortex. To help maintain the positioning of the capsules, a small piece of collagen was placed over the capsules and the incision sutured closed. Animals were then placed on a temperature-controlled pad until recovery from anesthesia. These procedures resulted in the formation of 3 experimental groups: (1) Stroke only (MCA+craniotomy but no transplant; N=10), (2) Stroke+control transplant (empty capsules; N=10) and (3) Stroke+choroid plexus loaded capsules; N=11).

Behavioral Testing

Motor Asymmetry

Because motor asymmetry (i.e., bias movements to one side of the body) is consistently displayed by MCA-occluded rats (Borlongan C. V., Cahill D. W., Sanberg P. R., “Locomotor and passive avoidance deficits following occlusion of the middle cerebral artery.” Physiol Behav. 58:909-17, 1995a.; Borlongan C. V., Martinez R., Shytle R. D., Freeman T. B., Cahill D. W., Sanberg P. R., “Striatal dopamine-mediated motor behavior is altered following occlusion of the middle cerebral artery.” Pharmacol Biochem Behav. 52:225-9, 1995b.) the elevated body swing test (EBST) was used to confirm the functional consequences of the MCA occlusion and to quantify improvements in motor function produced by the choroid plexus transplants. Animals were tested daily on days 1, 2, and 3 post surgery. Previous studies demonstrated that the EBST (Borlongan C. V., Cahill D. W., Sanberg P. R., “Locomotor and passive avoidance deficits following occlusion of the middle cerebral artery.” Physiol Behav. 58:909-17, 1995a.; Borlongan C. V., Martinez R., Shytle R. D., Freeman T. B., Cahill D. W., Sanberg P. R., “Striatal dopamine-mediated motor behavior is altered following occlusion of the middle cerebral artery.” Pharmacol Biochem Behav. 52:225-9, 1995b.; Borlongan C. V., Sanberg P. R., “Elevated body swing test: a new behavioral parameter for rats with 6-hydroxydopamine-induced hemiparkinsonism.” J Neurosci.15:5372-8, 1995c.) reliably detects stable motor asymmetry at these early time points. Individual animals were gently picked up at the base of the tail and elevated until the animal's nose was at a height of 5 cm above the test surface. The direction of the swing, either left or right, was counted once the animals head moved sideways approximately 10 degrees from the midline position of the body. After a single test, the animal was lowered and allowed to move freely for 30 seconds prior to retesting. These steps were repeated 20 times for each animal.

Neurological Evaluation

Animals were tested for neurological function using a conventional battery of tests (Bederson test). Each animal received a single test three days post surgery. A neurologic score for each rat was obtained using 3 tests that included (1) contralateral hindlimb retraction that measured the ability of the animal to replace the hindlimb after it was displaced laterally by 2 to 3 cm, graded from 0 (immediate replacement) to 3 (replacement after minutes or no replacement); (2) beam walking ability graded 0 for a rat that readily traversed a 2.5-cm-wide, 80-cm-long beam to 3 for a rat unable to stay on the beam for 10 seconds; and (3) bilateral forepaw grasp that measured the ability to hold onto a 2-cm-diameter wooden rod, graded 0 for a rat with normal forepaw grasping behavior to 3 for a rat unable to grasp with the forepaws. The scores from all 3 tests were conducted over a period of approximately 15 minutes and were combined to give an average neurologic deficit score (total score divided by three).

Histology

Following behavioral testing on day 3 post-stroke, animals were anesthetized with lethal dose of equithesin (500 mg/kg, i.p.), perfused with 100 mls of ice-cold saline, decapitated and the brains harvested. To confirm viability o f the transplanted cells the capsules were flushed from the transplant site using sterile saline. [state how cell viability determined-what dyes were used? Needs Steve's input here] Quantitative histological determinations of infarct volume were performed using standard TTC staining and quantitative image analysis as previously described (57). Infarct volume was determined using the following formula=2 mm (thickness of the slice)×[sum of the infarction area in all brain slices (mm²)].

In vitro Biological Activity

In vitro biological activity of the encapsulated choroid plexus was determined by placing conditioned media onto primary day 15 embryonic cortical neurons and measuring its effects on neuronal survival under serum deprivation conditions. The techniques used for preparing and maintaining primary cortical neuronal cultures were similar to those described previously (Fukuda A, Deshpande SB, Shimano Y, Nishino H. “Astrocytes are more vulnerable than neurons to cellular Ca2+ overload induced by a mitochondrial toxin, 3-nitropropionic acid.” Neuroscience. 87:497-507, 1998. ). Brains were removed from Wistar rats on embryonic day 15 and incubated in HBSS chilled on ice. The cortical tissues were dissected free, chopped into small pieces and incubated with Ca²⁺-free Hanks' solution containing trypsin (0.05 mg/ml) and collagenase (0.01 mg/ml) at 37° C. for 30 minutes, followed by the addition of soybean trypsin inhibitor (0.1 mg/ml) and DNase (0.1 mg/ml). The tissue was then centrifuged for 5 minutes (1000 rpm) in Dulbecco's modified Eagle's medium supplemented with 10% fetal bovine serum. The pellet was re-suspended and a homogenous cell suspension was made by gentle trituration using a fire-polished Pasteur pipette. Cells were plated on 35 mm tissue culture dishes (5×10⁴ cells/ml). The culture dishes were kept in a humidified incubator under 5% CO₂ and 95% air at 37° C. for 4 days. On day 4, cells were re-plated in 24-well plates, and over the next two days, a subset of cells were cultured without serum and with a range of concentrations of conditioned media (0-30%). On day 6, cell viability was analyzed using Trypan blue exclusion. All studies were conducted in triplicate.

### RESULTS

Encapsulation of Choroid Plexus

Statement of capsule size, appearance, approximate cell loading, viability before and after transplantation-include photomicrographs. Needs Steve's input here.

Behavioral Testing:

Choroid Plexus Grafts Reduce Stroke-Induced Motor Deficits

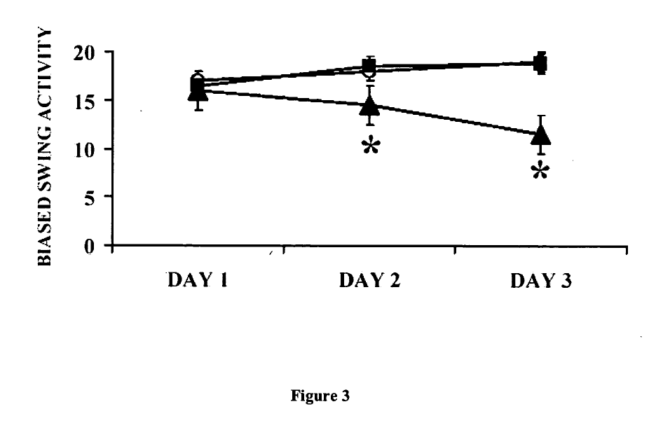

As shown in FIG. 3, choroid plexus transplants significantly reduced the motor asymmetry produced by MCA occlusion. An overall ANOVA revealed significant treatment effects over the 3 day post-stroke period (F_{2,90}=28.07, p<0.0001). While a trend towards improved performance was seen in those animals receiving choroid plexus transplants as early as 1 day post surgery this benefit was modest and did not reach statistical significance (p>0.05). Bonferroni's post-hoc t-tests did, however, demonstrate that stroke animals receiving choroid plexus transplants (▴)displayed significant ameliorations of motor asymmetry (FIG. 3) at days 2 and 3 post-surgery (>16% and >23%, respectively) compared to control animals (empty capsules (▪) or stroke only (∘); p's<0.01). These reductions translated to an average motor asymmetry of 74% and 62%, which are below the conventionally accepted 75% criterion for MCA-occluded rats to be considered significantly impaired on this test. No significant changes were noted in either control group throughout testing.

Choroid Plexus Grafts Reduce Neurological Deficits

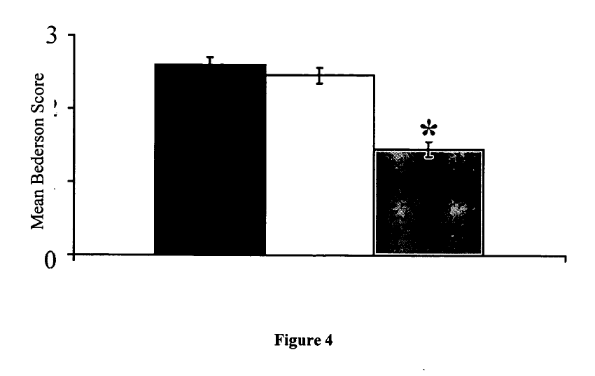

Similar benefits of encapsulated choroid plexus transplants were observed on neurologic impairment. Animals were tested for neurological function on day three post surgery using the Bederson test (ANOVA, F_{2,28}=50.6, p<0.0001) (FIG. 4). Post-hoc comparisons demonstrated that while MCA occlusion produced pronounced deficits in performance in control animals, stroke animals that received choroid plexus transplants exhibited significant improvements in neurological performance. Those animals receiving choroid plexus transplants were improved by 35%-40% relative to the control animals (p's <0.0001). There were no detectable differences in performance between the control groups at any time or on any test (p>0.10).

Histology

Choroid Plexus Grafts Reduce Stroke-Induced Cerebral Infarcts

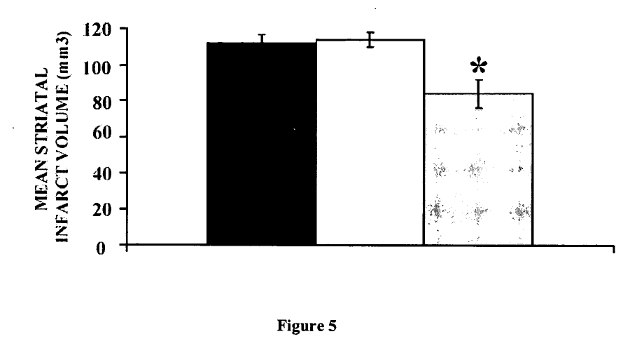

Three days following MCA occlusion and transplantation, the volume of cerebral infarct was determined in all animals using TTC staining and quantitative image analysis. Consistent with previous studies, MCA occlusion produced a large cerebral infarct that encompassed much of the striatum in control animals. The attenuation of behavioral deficits in stroke animals receiving choroid plexus transplants was accompanied by a significant reduction in cerebral infarction (ANOVA, F_{2,28}=4.77, p<0.05). Relative to control animals the volume of striatal infarct was significantly reduced by about 30% (FIG. 5; p's<0.05).

In vitro biological activity

Conditioned Media from Cultured Choroid Plexus Protects Neurons Against Serum Deprivation-Induced Cell Death

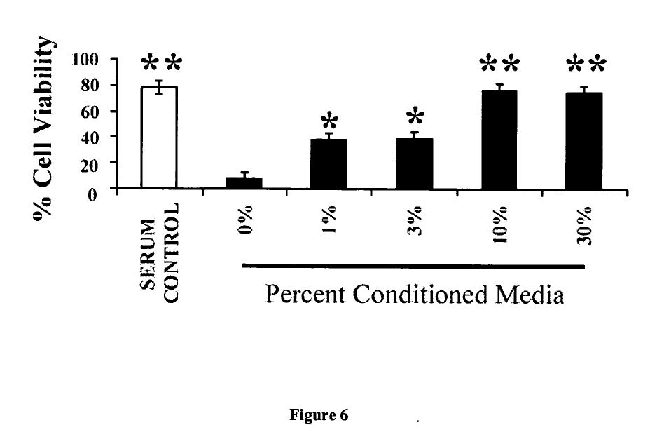

In vitro tests demonstrated that molecules secreted from the encapsulated choroid plexus exerted potent neurotrophic effects. An overall ANOVA revealed treatment effects on neuronal cell viability (ANOVA, F_{5,38}=109.01, p<0.0001). Primary cortical neurons deprived of serum for 2 days exhibited significant cell death (approximately 90%) compared to cells maintained in serum media (FIG. 6). Conditioned media collected from pig choroid plexus significantly protected against serum deprivation-induced cell death. This effect was dose-dependent with maximal effects obtained when serum-deprived neurons were cultured with 10% to 30% conditioned media from pig choroid plexus (p's <0.0001). At these concentrations, neuronal survival was 60%-85% and did not differ significantly from serum maintained cells (p's>0.05).

### DISCUSSION

The present set of experiments are the first to demonstrate the in vivo and in vitro neuroprotective effects of choroid plexus on neurons that are otherwise destined to die. In vitro, conditioned media from alginate-encapsulated choroid plexus produced a clear and dose-dependant protection of embryonic cortical neurons under conditions of serum deprivation. Parallel in vivo studies further demonstrated that transplanted choroid plexus significantly reduced the extent of cerebral infarction and motor/neurological deficits following MCA occlusion in rats. These studies did not attempt to optimize the transplant site or the numbers of cells used per recipient. Rather, based on previous studies, we simply placed the cell-loaded capsules on the cortex overlying the striatal region that would be normally infarcted following MCA occlusion. Without wishing to be bound, we believe this paradigm provided a fairly stringent test of the ability of the molecules secreted from the choroid plexus to exert a neuroprotective effect since the molecules would be required to diffuse from the capsules and through several mm of cortical tissue. Accordingly, the concentrations of therapeutic molecules reaching the infarcted region would be modest compared to those achieved locally. Nonetheless, even under these less than ideal conditions, a significant structural and functional benefit was produced by the choroid plexus transplants.

These studies used alginate microcapsules to encapsulate the choroid plexus The semipermeable membrane encapsulating, or surrounding the cells admits oxygen and required nutrients and releases bioactive cell secretions, but restricts passage of larger cytotoxic agents from the host immune defense system. The capsules provide the advantages of eliminating the need for chronic immunosuppression of the host and allowing the implanted cells to be obtained from xenogeneic sources (i.e. porcine cells used in the current studies) thus avoiding the constraints associated with cell sourcing. These microcapsules conferred the additional advantage of facilitating transplantation and localization on the cerebral cortex in the current studies.

8. Example 2

### MATERIALS AND METHODS

Animals

All procedures used in this study adhered to NIH and Society for Neuroscience guidelines for use of animals in research. All surgical procedures were conducted under aseptic conditions. All efforts were made to minimize animal suffering and to reduce the number of animals used. Adult male Wistar rats (supplied by University of Auckland, NZ) approximately 3 months of age and weighing 250-350 grams served as subjects. Animals were housed in a temperature (22+/−1° C.) and humidity (50+/−5%) controlled environment and had free access to food and water throughout the study, except for 4 hours prior to surgery.

Adult Rat and Neonatal Pig Choroid Plexus Cell Preparations.

Animals were anaesthetized with ketamine and xylazine, killed by exsanguination and the brain dissected and cut through the midline to reveal the fork of the choroid vessels. The choroid plexus was gently extracted and placed in Hanks Balanced Salt Solution (HBSS, 0-4° C.) supplemented with 2% human serum albumin. The tissue was chopped finely with scissors, allowed to settle and the supernatant removed. Collagenase (Liberase, Roche, 1.5 mg/ml, in 5 ml HBSS at 0-4° C.) was added and the chopped tissues mixed, allowed to sediment at unit gravity (at 1×g) and the supernatant removed. Collagenase (1.5 mg/ml, in 15 ml HBSS at 0-4° C.) was added and the preparation warmed to 37° C. and stirred for 15-20 minutes. The digested material was triturated gently with a 2 ml plastic Pasteur pipette, passed through a 200 um stainless steel filter.

Rat preparations were mixed with an equal volume of RPMI medium supplemented with 10% fetal bovine serum (Gibco). Neonatal pig preparations were mixed with an equal volume of RPMI medium supplemented with 10% neonatal porcine serum (prepared at Diatranz/LCT). The preparations were centrifuged (500 rpm, 4° C. for 5 minutes), the supernatant removed and the pellet gently resuspended in 30 ml RPMI supplemented with the appropriate serum. Microscopy revealed a mixture of epithelioid leaflets or clusters of cells, a bout 50-200 microns in diameter, and blood cells. Blood cells were removed by allowing the mixture to sediment at unit gravity for 25 minutes at 0-4° C., removing the supernatant and resuspending.

The preparation was adjusted to approximately 30,000 clusters/ml in RPMI with 10% serum and placed in non-adherent Petri dishes. Half of the media was removed and replaced with fresh media (5ml) after 24 h and again after 48 h. By this time most clusters assumed a spherical, ovoid or branched appearance. Before encapsulation the clusters were washed by sedimenting 3×in 2% human serum albumin (30 ml) at room temperature.

Surgery

Immediately prior to surgery, rats were anesthetized with equithesin (300 mg/kg; i.p.) and positioned in a stereotaxic instrument (Kopf, Tujunga Calif.). A midline incision was made in the scalp and a hole drilled through the skull for placement of a cell-loaded alginate capsules into the striatum using an 18-guage Teflon catheter mounted to the 15 stereotaxic frame. The stereotaxic coordinates for implantation were: 0.5 mm anterior to Bregma, 1.5 mm lateral to the sagittal suture, and 7.5. mm below the cortical surface. Following implantation, the skin was sutured closed.

Three days following implantation of the capsules, all animals were anesthetized, placed into the stereotaxic instrument and unilaterally injected with 225 nmol of QA (Sigma) into the striatum at the following coordinates: 1.2 mm anterior to Bregma, 2.6. mm lateral to the sagittal suture, and 5.5 mm ventral to the surface of the brain. QA was infused into the striatum using a 28-gauge Hamilton syringe in a volume of 1 μ/g over 5 minutes. The injection cannula was left in place for an additional 2 minutes to allow the QA to diffuse from the needle tip, after which the cannula was removed, and the skin sutured closed. Control animals received either no capsule or capsules containing mock-transfected cells. This resulted in the formation of 4 experimental groups:

1. QA lesion, no capsules (N=8) 2. QA lesion, empty capsules (N=8) 3. QA lesion, rodent choroid plexus (N=8) 4. QA lesion, porcine choroid plexus (N=8)

Immediately following the QA lesion, animals were injected i.p. with 10 mL of a lactated Ringer's solution. Twenty eight days post capsule implantation, animals were anesthetized and decapitated and the brains processed for histology.

Behavioral Testing

Placing: To quantify potential sensory neglect, the forelimb placing test was used to test the animal's ability to make directed forelimb movements in response to sensory stimuli. Rats were held so that their limbs were hanging unsupported and the length of their body was parallel to the surface of a stainless steel table. They were raised to the side of the table so that their whiskers made contact with the top surface on 10 trials for each forelimb. Rats were given one trial 14 days post-lesion.

### RESULTS

Behavioral Testing

Intrastriatal injections of QA produced significant performance deficits in the placement, bracing, and akinesia tests. This was evidenced by a decrease of 90% in the number of placements taken relative to the unimpaired limb in animals receiving QA plus no capsule implant. No change in performance was noted in animals receiving control implants (empty capsules) although a slight increase in the number of placements compared to animals receiving QA plus no implant was noted. In contrast a marked behavioral protection was observed when encapsulated choroid plexus cells were implanted immediately adjacent to the QA-lesioned striatum. Relative to the normal limb, performance of the impaired limb was completely normalized as assessed using this measure (see Table 1). No differences were noted in the behavioral protection produced by rodent choroid plexus compared to porcine choroids plexus.

TABLE 1
Behavioral Assessment Following Choroid Plexus Transplants

| Treatment Group | Places |
|---|---|
| Lesion (no capsule) | |
| intact limb | 9.0 |
| impaired limb | 1.0 |
| Lesion + empty capsule | |
| intact limb | 9.5 |
| impaired limb | 3.0 |
| Lesion + rodent choroid plexus | |
| intact limb | 9.0 |
| impaired limb | 9.0 |
| Lesion + porcine choroid plexus | |
| intact limb | 9.0 |
| impaired limb | 9.0 |

In conclusion, we report here for the first time that transplanted choroid plexus has robust neuroprotective effects in a rodent model of acute stroke and a rodent model of Huntington's disease, providing support for the use of choroid plexus as a source for cell-based delivery of growth factors and/or cell replacement therapy across a range of acute and chronic CNS diseases.

What we claim:

1. A treatment species for administration to a mammalian recipient comprising or including one or more of:
a) a cell population capable of producing one or more factors, or
b) a cell culture capable of producing one or more factors or
c) conditioned media from a cell culture containing one or more factors.

2. A treatment species for administration to a mammalian recipient as claimed in claim 1 wherein the treatment species is capable of releasing or administering to the recipient, a secretion derived from the cells, more preferably the secretion includes cell derived factors.

3. A treatment species for administration to a mammalian recipient as claimed in claim 1 wherein the factors are neurotrophins, growth factors, matrix cell support factors, proteases capable of degrading toxic protein precipitates (such as amyloid and huntingtin), and proteins capable of complexing toxic metal ions (such transferrin and ceruloplasmin).

4. A treatment species for administration to a mammalian recipient as claimed in claim 1 wherein the cells are derived from the embryonic neural crest.

5. A treatment species for administration to a mammalian recipient as claimed in claim 1 wherein the cells are selected from one or more choroid plexus cells or one or more glial or glial-derived cells or epithelial cells.

6. A treatment species for administration to a mammalian recipient as claimed in claim 5 wherein the cells have been subject to genetic modification.

7. A treatment species for administration to a mammalian recipient as claimed in claim 1 wherein the cells are choroid plexus cells and the treatment species is capable of releasing or administering to the recipient, a choroid plexus derived secretion.

8. A treatment species for administration to a mammalian recipient as claimed in claim 7 wherein the choroid plexus derived secretion includes choroid plexus derived factors.

9. A treatment species for administration to a mammalian recipient as claimed in claim 1 wherein the cells are living cells.

10. A treatment species for administration to a mammalian recipient as claimed in claim 1 wherein the treatment species is derived from one or more choroid plexus cells obtained or derived from a donor mammalian species.

11. A treatment species for administration to a mammalian recipient as claimed in claim 10 wherein the treatment species is derived from a cell culture.

12. A treatment species for administration to a mammalian recipient as claimed in claim 11 wherein the cell culture is a primary culture and/or a secondary culture and/or comprises cell lines derived from choroid plexus cells including immortalized cell lines.

13. A treatment species for administration to a mammalian recipient as claimed in claim 1 wherein the cell population or cell culture is from a donor mammalian species which is a different species to the recipient.

14. A treatment species for administration to a mammalian recipient as claimed in claim 13 wherein the donor mammalian species is a pig, rabbit or rat.

15. A treatment species for administration to a mammalian recipient as claimed in claim 14 wherein the donor mammalian species is a virus-free neonatal pig, rabbit or rat.

16. A treatment species for administration to a mammalian recipient as claimed in claim 1 wherein the cell population or cell culture is from a donor mammalian species which is the same species as the recipient.

17. A treatment species for administration to a mammalian recipient as claimed in claim 16 wherein the donor mammalian species is human.

18. A treatment species for administration to a mammalian recipient as claimed in claim 1 wherein the treatment species may comprise or include one or more choroid plexus cells.

19. A treatment species for administration to a mammalian recipient as claimed in claim 1 wherein the treatment species may comprise or include one or more choroid plexus cells encapsulated in a suitable encapsulation medium.

20. A treatment species for administration to a mammalian recipient as claimed in claim 19 wherein the encapsulation medium is an alginate.

21. A treatment species for administration to a mammalian recipient as claimed in claim 1 wherein the treatment species may comprise or include one or more naked choroid plexus cells.

22. A treatment species for administration to a mammalian recipient as claimed in claim 1 wherein the treatment species may comprise or include one or more choroid plexus cells contained within a confinement means.

23. A treatment species for administration to a mammalian recipient as claimed in claim 22 wherein the confinement means is factor permeable in vivo.

24. A treatment species for administration to a mammalian recipient as claimed in claim 1 wherein the treatment species may comprise or include one or more isolated choroid plexus cells.

25. A treatment species for administration to a mammalian recipient as claimed in claim 1 wherein the treatment species may comprise or include media harvested from choroid plexus cells (whether these be naked, isolated, cultured, modified or otherwise).

26. A treatment species for administration to a mammalian recipient as claimed in claim 1 wherein the treatment species may comprise or include one or more choroid plexus cells and/or the media harvested from one or more choroid plexus cells (whether these be naked, isolated, cultured, modified or otherwise), in a pump or implantable infusion device.

27. A treatment species for administration to a mammalian recipient as claimed in claim 1 wherein the treatment species may comprise or include one or more choroid plexus cells and/or the media harvested from one or more choroid plexus cells (whether these be naked, isolated, cultured, modified or otherwise), in a bio-erodable polymer.

28. A treatment species for administration to a mammalian recipient as claimed in claim 1 wherein the treatment species may comprise or include cerebrospinal fluid containing one or more choroid plexus cells and/or the secretion from one or more choroid plexus cells obtained from the recipient, or another mammalian species.

29. A method of preparing a treatment species for administration to a recipient mammal comprising or including the steps of:
a) obtaining one or more cells capable of producing one or more factors, from a donor species
b) reparing the treatment species.

30. A method of preparing a treatment species for administration to a recipient mammal as claimed in claim 29 wherein the one or more cells are choroid plexus cells.

31. A method of preparing a treatment species for administration to a recipient mammal as claimed in claim 29 wherein the method comprises or includes the steps:
1) obtaining one or more choroid plexus cells from a donor species;
2) culturing the one or more choroid plexus cells;
3) preparing the treatment species.

32. A method of preparing a treatment species for administration to a recipient mammal as claimed in claim 31 wherein step 1) of obtaining the one or more choroid plexus cells from the donor species comprises or includes obtaining the fresh tissue from the donor and dissociating the tissue mechanically and/or by enzymatic digestion.

33. A method of preparing a treatment species for administration to a recipient mammal as claimed in claim 31 wherein step 2) of culturing the one or more choroid plexus cells comprises or includes preparing the cells in such a way as to produce choroid plexus cell clusters of a regular size (preferably between 50-300 microns in diameter).

34. A method of preparing a treatment species for administration to a recipient mammal as claimed in claim 31 wherein the step 3) of preparing the treatment species may comprise or include one of the following:
encapsulation of the cells, or media obtained therefrom in a suitable encapsulation medium;
confinement of the cells, or media obtained therefrom in a suitable confinement means;
housing of the cells, or media obtained therefrom in a pump or implantable infusion device;
housing of the cells, or media obtained therefrom in a bioerodable polymer;
addition of the cells or media obtained therefrom to a pharmaceutically acceptable diluent and/or excipient and/or carrier.

35. A method of preparing a treatment species for administration to a recipient mammal as claimed in claim 34 wherein the encapsulation medium is an alginate.

36. A treatment species prepared according to the method of claim 34.

37. A method of administering a treatment species to a recipient comprising or including:
preparation of a treatment species as in claim 31,
administering the treatment species to a targeted area of the recipient.

38. A method of administering a treatment species to a recipient as claimed in claim 37 wherein the administration of the treatment species to the recipient results in one or more of the following events:
treatment of cells of the nervous system damaged by events such as injury, disease, trauma;
protection against damage to cells of the nervous system arising from future events as injury, disease, trauma;
prevention or minimisation of apoptotic nervous system cell death;
regeneration of damaged cells of the nervous system;
impeding or stopping cell death cascades resulting from events such as nervous cell injury, disease, trauma.

39. A method of administering a treatment species to a recipient as claimed in claim 37 wherein the cells are in the central nervous system.

40. A method of administering a treatment species to a recipient as claimed in claim 39 wherein the cells are in the brain.

41. A method of administering a treatment species to a recipient as claimed in claim 37 wherein the cells are in the peripheral nervous system.

42. A method of administering a treatment species to a recipient as claimed in claim 37 wherein the administration of the treatment species results in one or more of the following:
treatment or prevention of a neurodegenerative disease;
repair of damage caused by acute trauma to the brain;
treatment of damage resulting from pre-birth asphyxia;
treatment of damage resulting from neonatal ischemia (pre, during, post birth);
treatment of infection related cell death (including from meningitis and
encephalitis);
treatment of damage resulting from pressure related cell death (such as resulting from head injury to the recipient);
treatment of auto-immune disorders, including for example, demyelinating conditions (such as multiple sclerosis); rheumatoid
arthritis, crohn's disease, ulcerative colitis;
Treatment of sense loss due to apoptotic events, such as RP, diabetic retinopathy, macular degeneration, optic nerve damage;
Treatment of inborn errors of metabolism that mostly affect the central nervous system.

43. A method of administering a treatment species to a recipient as claimed in claim 37 wherein the step of administering the treatment species to a targeted area of the recipient includes one or more of the following:
administering the treatment species into the central nervous system;
administering the treatment species to a region outside but adjacent or proximal the central nervous system;
administering the treatment species directly into the region of the recipient which has suffered damage;
administering the treatment species to a region outside but adjacent or proximal the region of the recipient which has suffered damage;
administering the treatment species into the brain parenchyma;
administering the treatment species into the recipient so as to selectively target apoptotic cells; more preferably this comprises administering the treatment species into the margin of the damaged region;
administering the treatment species into the recipient so as to selectively target necrotic cells; more preferably this comprises administering the treatment species into the central aspect of the damaged region;
administering the treatment species into the ventricle;
administering the treatment species via lumbar puncture;
administering the treatment species into a CSF containing region.

44. A method of administering a treatment species to a recipient as claimed in claim 37 wherein the step of administering the treatment species to a targeted area of the recipient comprises or includes any administration so as to expose the targeted area to choroid plexus derived secretion.

45. A method of administering a treatment species to a recipient as claimed in claim 44 wherein the choroids plexus derived secretion includes or comprises choroids plexus derived factors.

46. A method of administering a treatment species to a recipient as claimed in claim 37 wherein the step of administering the treatment species to a targeted area of the recipient comprises or includes one or more of:
administration resulting in substantially immediate delivery of the treatment species to a targeted area; or
administration resulting in controlled delivery of the treatment species to the targeted area over a pre-selected time period.

47. A method of administering a treatment species to a recipient as claimed in claim 46 wherein the pre-selected time period is greater than five minutes.

48. A method of administering a treatment species to a recipient as claimed in claim 37 wherein the method includes one or more steps of:
suppressing the immune response of the recipient, more preferably by administration of immunosuppressive agents or drugs;
cooling the recipient;
Administering the cell preparation via a cannulated blood vessel.

49. A method of preventing, treating and/or ameliorating a neurological injury, disease or imbalance, comprising or including:
1) preparing an implant
2) implanting the implant in or around the central nervous system
wherein said implant results in, directly or indirectly, a beneficial effect on said neurological injury.

50. A method of preventing, treating and/or ameliorating a neurological injury, disease or imbalance as claimed in claim 49 wherein the implant consists of conditioned media.

51. A method of preventing, treating and/or ameliorating a neurological injury, disease or imbalance as claimed in claim 49 wherein the implant consists of living cells formed from the group of cells including choroid plexus cells, glial derived cells and neurons.

52. A method of preventing, treating and/or ameliorating a neurological injury, disease or imbalance as claimed in claim 49 wherein said living cells are formed from a homogeneous mix of cell populations.

53. A method of preventing, treating and/or ameliorating a neurological injury, disease or imbalance as claimed in claim 49 wherein the cells are encapsulated in a biocompatible medium.

54. A method of preventing, treating and/or ameliorating a neurological injury, disease or imbalance as claimed in claim 49 wherein the implant is implantable into a localised area in or around the central nervous system proximate to the neurological injury and/or into supporting structures of the central nervous system.

55. An implant for implantation into the central nervous system and/or surrounding supporting structures of a mammalian recipient,
wherein the said implant consists of conditioned media and/or living cells formed from a homogeneous mix of cell populations.

56. An implant for implantation into the central nervous system and/or surrounding supporting structures of a mammalian recipient as claimed in claim 55 wherein said implant consists of one or more living cells formed from a group of cells including choroid plexus cells, glial derived cells and neurons.

57. A surgical method for treatment of a human comprising or consisting the following steps:
1) accessing through the skull and dura mater
2) administering an implant as described previously into a cerebral fluid filled space.

58. A surgical method for treatment of a human as claimed in claim 57 wherein step 2 comprises or includes administering the implant directly into the brain parenchyma.

59. A surgical method for treatment of a human as claimed in claim 57 wherein step 2 comprises or includes administering the implant external to the brain parenchyma.

60. A surgical method for treatment of a human as claimed in claim 57 wherein the implant is located subdurally but still external to the brain parenchyma.

61. A pharmaceutical composition for treatment or prevention of a disease or condition in a mammal in need of treatment by therapeutic administration of an implant comprising:
1) one or more living cells capable of producing one or more factors,
2) at least one permeation-enhancement agent for transmucosal drug uptake.

62. A pharmaceutical composition for treatment or prevention of a disease or condition in a mammal as claimed in claim 61 wherein the one or more living cells include one or more choroid plexus cells.
